(19) United States
(12) Patent Application Publication
(10) Pub. No.: US 2017/0196757 A1
(43) Pub. Date: Jul. 13, 2017

(54) Treatment of neuropathic pain using low pressure suction apparatus

(71) Applicant: HLD Healthy Life Devices Oy, Espoo (FI)

(72) Inventors: Kalle Palomäki, Helsinki (FI); Steven Basta, Menlo Park, CA (US)

(73) Assignee: HLD Healthy Life Devices Oy, Espoo (FI)

(21) Appl. No.: 15/401,267

(22) Filed: Jan. 9, 2017

Related U.S. Application Data
(60) Provisional application No. 62/275,811, filed on Jan. 7, 2016.

(51) Int. Cl. A61H 9/00; A61M 1/08

(52) U.S. Cl. CPC A61H 9/0057; A61H 9/0007; A61M 1/08; A61H 2201/5007

(57) ABSTRACT

A method for at least temporarily alleviating neuropathic pain comprising obtaining a preferably both electrically powered and portable treatment apparatus capable of contacting, via a treatment head, a target area of a patient and introducing low-pressure suction, i.e. negative pressure flow, thereto, configuring the treatment apparatus so as to introduce predefined, advantageously, pulsating pressure variation to a suction opening arranged at the treatment head, and applying the suction opening of the treatment head on the target area while the treatment apparatus is on and providing the suction effect corresponding to the configuration.

### PRIORITY

This application claims priority of U.S. provisional application No. 62/275,811 which was filed on Jan. 7, 2016 and the contents of all of which are incorporated herein by reference.

### FIELD OF THE INVENTION

The present invention generally relates to medical methods. Especially, however not exclusively, the invention pertains to treatment of neuropathic pain utilizing an apparatus producing low pressure suction on a target area of a patient.

### BACKGROUND

Neuropathic pain refers to a chronic pain state in which the pain is caused by damage or disease affecting the somatosensory nervous system. Neuropathic pain can be associated with abnormal sensations called dysesthesia or pain from normally non-painful stimuli.

The pain experienced by the person suffering from neuropathic pain can be continuous pain or/and episodically occurring shooting-, burning-, stabbings- or electric shocks-like pain. Symptoms can also include tingling and/or numbness.

Neuropathic pain typically results from a disorder of the central or peripheral nervous system. Neuropathic pain can thus be divided into central, peripheral or mixed neuropathic pain. Typical causes for central neuropathic pain are spinal cord injury, spinal disc herniation, multiple sclerosis or strokes. Peripheral neuropathic pain is typically caused by herpes zoster infection, HIV-associated neuropathy, nutritional deficiencies, toxins, remote manifestations of malignancies, immune mediated disorders, diabetes, alcoholism, amputation (phantom limb pain) and physical trauma to a nerve trunk. Fibromyalgia is a condition causing pain resembling neuropathic pain. Diabetic neuropathy is one of the common forms of neuropathy due to a large number of patients. In case of cancer, a tumor can compress a nerve causing neuropathic pain. Chemotherapy is also known to cause so called chemotherapy-induced peripheral neuropathy. There are also mixed type of neuropathic pain such as postherpetic neuralgia caused by varicella zoster virus.

Commonly used treatment for neuropathic pain is medication. These include use of, e.g., anticonvulsants, antidepressants, botulinum toxin type A, cannabinoids, neuromodulators, NMDA (N-Methyl-_{D}-aspartate) antagonists and dietary supplements.

Medication often has side effects as is well-known. The side effects can become worse in case the medication is continued to be used for a long period of time. In case of stronger pain relievers, such as opioids, the patient may develop an addiction to the medicine. In some cases new medication is prescribed to alleviate the side effects of the earlier medicine which as a combination can cause new symptoms.

There are also other types of treatment such as invasive/implantable methods including transcutaneous electrical nerve stimulation or spinal cord stimulator. Invasive methods naturally pose known risks, same as any surgical procedure. Implantable devices are foreign objects to patient's body and pose the risk of rejection and rejection-related symptoms.

Relief to the neuropathic pain can also be gained from physical therapy, working with a counsellor, relaxation therapy, acupuncture, and massage therapy.

Use of a combination of various treatments is also common in treating neuropathic pain. Typically still just a portion of patients benefit from the abovementioned forms of treatment.

### SUMMARY

The objective is to provide a method for treating neuropathic pain so as to provide at least temporary relief from the symptoms thereof while still preferably alleviating or overcoming one or more problems of the various prior art solutions.

Therefore, according to an embodiment, a method for at least temporarily alleviating neuropathic pain comprises
obtaining a preferably both electrically powered and portable treatment apparatus capable of contacting, via a treatment head, a target area of a patient and introducing low-pressure suction, i.e. negative pressure flow, thereto,
configuring the treatment apparatus so as to introduce predefined, advantageously, pulsating pressure variation to a suction opening arranged at the treatment head, and
applying the suction opening of the treatment head on the target area while the treatment apparatus is on and providing the suction effect corresponding to the configuration.

In various embodiments, said configuring may include determination of at least one feature selected from the group consisting of: size of the opening at the treatment head, shape and/or dimensions of a treatment cup located at the treatment head, used treatment cup among multiple options of different material, shape, opening and/or dimensions, pulse duration, duty cycle, signal period, signal (repetition) frequency, pulse pressure, minimum, maximum or optimum duration of subjecting the treatment head to an area at a time, and minimum, maximum, or optimum duration of a treatment session. The beginning or end of any aforesaid duration may be optionally indicated audibly, tactilely (e.g. vibration) and/or visually to the patient or other operator of the apparatus during use for guidance.

In various embodiments, the apparatus may incorporate a central unit and a functionally connected treatment head that is preferably configured as hand-held by the patient or other operator of the apparatus. The connection may be established via a hose between the unit and head. The internal wall of said hose may define an air duct between a pressure chamber of the central unit and opening of the treatment head preferably provided by a treatment cup of the head. Further, an electrical connection between the two may be established via electrical wiring or wirelessly e.g. via electromagnetic coupling. The hose may transport gaseous matter, typically air, and thus cause the pressure pulsation (or a continuous low pressure suction) due to a pulsating pumping action executed by pumping mechanism of the central unit. The central unit may include a vacuum pump for the purpose. In some embodiments, a fan could be alternatively utilized.

In various embodiments, said applying incorporates maintaining the portion, such as a cup, of the treatment head defining the opening substantially in contact, typically skin contact, with location at least partly defining the target area for a predefined time period, which may optionally refer to duration of few seconds, or e.g. about 3-5 pulsations.

Thereafter, the head may be re-positioned to a new location that may optionally overlap with the previous one. Such procedure may be continued until the target area as a whole has been treated at least once, treating at least at about three to five different spots. In some embodiments, the treatment may be alternately directed to the same locations constituting the target area, e.g. as a repeated treatment pattern of several (sub-)areas with potentially overlapping portions, until a predefined overall period set for a treatment session, for instance, has lapsed.

The application technique in terms of motion may include stationary treatment. In stationary treatment, a certain target area or location, i.e. ‘sub-area’ therewithin in case the overall target area is too large for treatment by the treating head at a time (very typical scenario), is subjected to the low pressure suction treatment at a time by maintaining the treatment head thereon for some time, e.g. the aforementioned period of few seconds or few pulsations, prior to switching over to a next location. The switch over thus involves lifting the treatment head first away from the skin contact prior to moving it.

Alternatively or additionally, a so-called lift&twist type technique may be utilized. While a certain location is treated and the treatment head is lifted from the skin, simultaneous rotating, or ‘twisting’, action is performed.

Alternatively or additionally, substantially continuous sliding type treatment technique may be applied by moving the treatment head over the locations of the target area while maintaining the contact of the treatment head. As one variation, ‘knitting’ style sliding with a twist motion may be tried.

As a further option, a hybrid approach may be selected implying keeping the treatment head stationary relative to one location accommodating the treatment head while during the switchover to a next location the contact is still maintained instead of lifting the head away. As being mentioned above, the subsequent location may overlap with the previous one.

In various embodiments where switchover between the locations of stationary treatment occurs, at least the two subsequent areas may have some overlap, e.g. about 20-50% overlap.

The utility of the present invention arises from a variety of factors depending on each particular embodiment thereof. In many cases even substantially immediate relief of the symptoms of the neuropathic pain, such as reducing pain in patients with diabetic neuropathy, chemotherapy-induced neuropathy, fibromyalgia or Lyme disease, may be generally obtained by the suggested therapeutic method applying a low pressure suction apparatus. The reduction of pain in a patient may facilitate reduction in pain medication use. The generated pulsation generally stretches and generally mobilizes the skin, therefore stretching fascia and affecting the related structures of myofascia (i.e. soft tissue manipulation), thus typically making additional clearance below it and reducing the related pressure subjected to nerves, tissues, organs, veins, lymphatic vessels, stimulating mechanoreceptors, etc., while further activating them and e.g. the lymphatic system in general. The treatment may also yield various other advantageous effects described herein. In addition to negative/low pressure, i.e. suction, the pulsation may introduce certain amount of positive pressure to the tissues considering e.g. the areas opposite or adjacent to the target area under suction, or even to the target area itself due to the undulating nature of the pulsation and resulting skin motion. Accordingly, some benefits of positive pressure may be realized as well.

Yet, the condition may with some patients improve also in a longer term, even permanently, although in most cases, the suggested treatment is preferably given more or less regularly, e.g. repeated once a week or every second week, to ascertain the permanency of the effect thereof.

The embodiments of the present invention are considered both non-invasive and safe producing mainly mechanical effect on patient's tissue. The apparatus or generally equipment used to execute the treatment is affordable, portable, reliable, quiet and easy to service or use either by a patient or separate operator. The user, either being the patient himself/herself or dedicated operator, is not required to take extensive training to be able to apply the apparatus although at least basic understanding of human lymphatic system and operation of the apparatus is naturally considered advantageous in favor of the effectiveness of the therapy.

Accordingly, the therapy may be provided flexibly at different premises such as the home of a patient, at a physical therapist, or at some other desired location. There is no need to visit a doctor, hospital or some specialized therapy center to receive the treatment, which may facilitate the life of the patient considerably in terms of reduced travelling and associated cost, and gained time savings among other factors.

Finally, use of the apparatus is practically completely painless as the pulsation pressure may be adjusted to suit each use scenario such that the effect of the treatment is achieved while the suction effect remains moderate only causing gentle draft and pull type stretch sensations on the skin in contrast to different prior art methods and apparatuses, the effect of which is at least partly based on harsh, mechanical skin stretching and actually pinching activity due to the use of gripping elements such as rollers, which may be rather painful on sensitive swollen and potentially infected skin.

Relying on an apparatus according to an embodiment of the present invention may in many cases turn out advantageous also to the operator in contrast to e.g. manual therapy. The elbows, wrists, and hands of the operator may be spared from fatigue and pain, which are commonly induced by lengthy days of manual therapy.

Various other advantages will become clear to a skilled person based on the following detailed description.

The expression “a number of” refers herein to any positive integer starting from one (1), e.g. to one, two, or three.

The expression “a plurality of” refers herein to any positive integer starting from two (2), e.g. to two, three, or four.

The terms “first” and “second” do not denote any order, quantity, or importance, but rather are used to distinguish one element from another.

The exemplary embodiments of the invention presented in this patent application are not to be interpreted to pose limitations to the applicability of the appended claims. The verb “to comprise” is used in this patent application as an open limitation that does not exclude the existence of also un-recited features. The features recited in depending claims are mutually freely combinable unless otherwise explicitly stated.

The novel features which are considered as characteristic of the invention are set forth in particular in the appended claims. The invention itself, however, both as to its construction and its method of operation, together with additional objectives and advantages thereof, will be best understood from the following description of specific embodiments when read in connection with the accompanying drawings.

### BRIEF DESCRIPTION OF THE DRAWINGS

FIG. 1 illustrates schematically an embodiment of a treatment apparatus in accordance with the present invention.

FIG. 2 illustrates schematically an embodiment of a treatment apparatus in accordance with the present invention.

FIGS. 3A and 3B illustrate embodiments of a treatment cup of the treatment head in connection with the treatment apparatus in accordance with the present invention.

FIGS. 4A and 4B illustrate embodiments of a sealing part of the treatment head in accordance with the present invention.

FIG. 5 depicts schematically a use scenario of low pressure suction apparatus in treating a leg of a patient and an example of how the low pressure suction affects the target tissue.

FIG. 6 is a flow diagram disclosing an embodiment of a method in accordance with the present invention.

### DETAILED DESCRIPTION

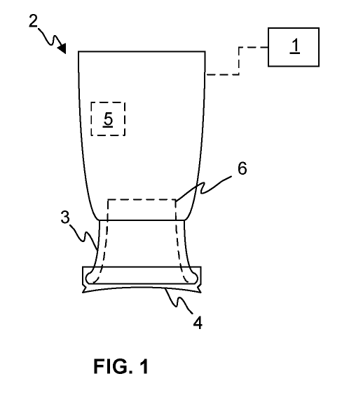

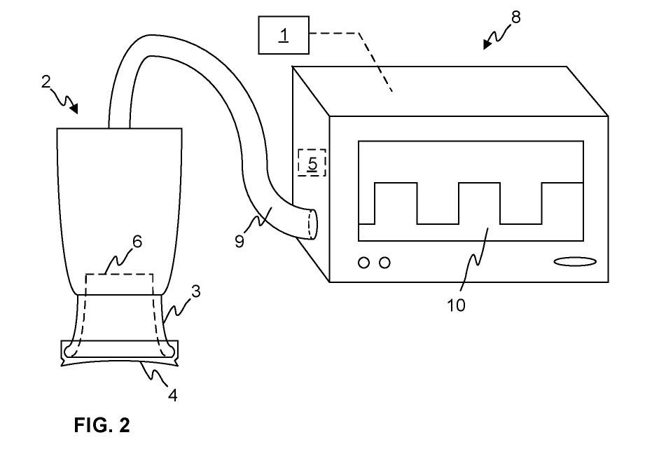

With reference to FIGS. 1 and 2, two embodiments of a treatment apparatus, or ‘device’, for use in connection with a therapeutic method in accordance with the present invention are illustrated.

Embodiments of the treatment apparatus comprise a treatment head 2, a central processing unit 1, a treatment cup 3, a sealing part 4, low pressure chamber 6 and means for producing the low pressure in the low pressure chamber 5. The central processing unit 1 may be arranged in connection with the treatment head 2 or in connection with a central unit 8 as shown in FIG. 2. The low pressure chamber 6 and the means for producing low pressure in the low pressure chamber 5 are shown schematically by dashed lines in FIGS. 1 and 2. The means for producing the low pressure 5 (negative pressure flow/suction effect), e.g. with a vacuum pump or a fan, may be in the treatment head 2 or in the central unit 8 in which case the means for producing the low pressure 5 is arranged in connection with the treatment head 2, e.g., by a hose 9. There may be a display in the treatment head 2 and/or 10 in the central unit 8. The central unit 8 may further incorporate an interface for an external display. In some embodiments, the features of a central unit 8 could even be combined with the treatment head 2 into an integral apparatus. In some embodiments, the pump or fan 5 could be included in the treatment head 2 while there is still provided a central unit 8 or similar element connected to the head 2 by a flexible element such as a cable or wiring for electricity and/or data transfer, for example. These types of embodiments would not obviously necessitate the use of a hose 9 for the suction flow between the elements 2, 8. When the pump or fan 5 is located within a common housing with the chamber 6, these two may still be connected via internal piping, hose(s) and/or duct(s), for example, for conveying the low pressure flow.

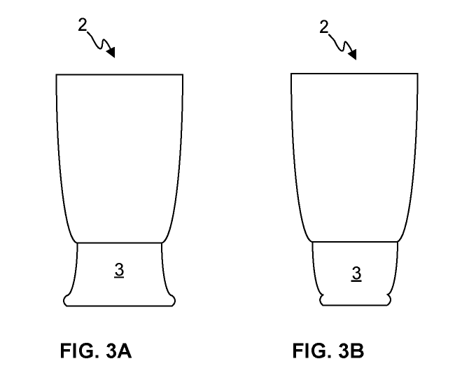

FIGS. 3A and 3B illustrate two embodiments of the treatment cup 3, one end of which is arranged to be in connection with the low pressure chamber 6 and the other end of which is arranged to be pressed towards the skin tissue (meaning in this application a degree of contact or a magnitude of force from a gentle touch/contact to more intense pressing) to direct low pressure suction to the target area, typically skin tissue, thus producing a bulge in the skin tissue. The skin preferably refers herein to all skin layers i.e. epidermis, dermis and hypodermis or subcutis (including superficial fascia).

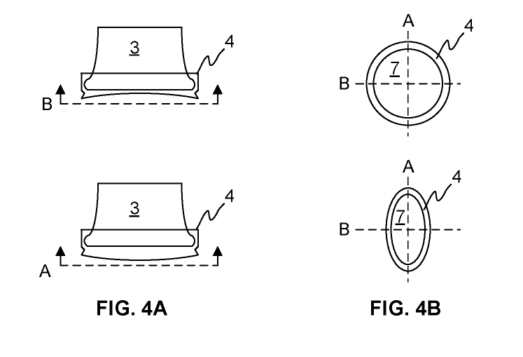

In FIGS. 3A and 3B, the end of the treatment cup 3 that is arranged to be pressed towards (typically against) the skin tissue is the lower end of the treatment cup 3. The treatment cup 3 is arranged to form a suction opening 7 (shown in FIG. 4B). FIGS. 3A and 3B illustrate two different shapes of treatment cups. There may also be other shapes and, thus, the examples in FIGS. 3A and 3B are not shown to pose limitations to the treatment apparatus utilizable in connection with the method according to the present invention. The treatment cup 3 with an appropriate size, shape and/or material may be chosen based on the nature or size of the target area of the skin to be treated, for instance.

The treatment cup 3 may further comprise a sealing part 4 advantageously made of a flexible material and arranged at the end of the treatment cup 3 that is arranged to be pressed against the skin tissue and to adapt to the shape of the suction opening 7 formed by the treatment cup 3 and to seal the gap between the end of the treatment cup 3 and the skin tissue when the end of the treatment cup 3 is pressed against the skin tissue.

The sealing part 4 may be made of any suitable flexible material, such as polyurethane or elastomeric material. The use of other plastic materials and silicon material is also possible. At the sealing part 4, it is naturally also possible to use a suitable material that reduces or increases the friction between the sealing part 4 and the skin tissue, and/or a material that improves the sealing, depending on whether the objective is to achieve a good mobility for the treatment head or as high a friction force as possible. Mobility may also be increased by using a number rotating elements, such as rollers, which are in connection with the treatment cup 3. The rotating elements, in addition to the sealing part 4, may also provide sealing function between the skin and the treatment cup 3.

As the treatment head 2 of the treatment apparatus is moved against the patient's skin, most comfortably using a handle, the effect of the low pressure causes a fold of skin to be pulled up into the low pressure chamber 6.

FIGS. 4A and 4B illustrate the shape of the sealing part 4 by side views from two substantially perpendicular directions. The sealing part 4, which is to be pressed against the skin tissue, is arranged to be concave in the direction of at least one of the main axes A or B of the suction opening 7 formed by the treatment cup 3.

In an embodiment of the treatment cup 3 of the treatment apparatus shown in FIG. 4A, the surface of the sealing part 4 to be pressed against the skin tissue is concave in the direction of the main axis B of the suction opening 7 formed by the treatment cup 3. The shape of the suction opening 7 may be elliptical or oval or round (some examples are shown in FIG. 4B). However, these are not the only feasible embodiments but other shapes may be used as well.

In the above examples, the sealing part 4 is shown as a separate part fastened to the end of the treatment cup 3. The sealing part 4 may be a disposable part, which is detached after use and replaced by a new one in the beginning of the treatment of a new patient. Alternatively, the sealing part 4 may be reusable after washing or disinfecting. The fastening to the end of the treatment cup 3 may be achieved by means of various connections. At the end of the treatment cup 3, which is to be pressed against the patient's skin, there may be a flange in the radial direction, which is used for fastening the sealing part 4 made of a flexible material to its place. Thus, the schematically shown connection of the sealing part 4 in figures is not to be understood as a factor limiting the embodiments of the treatment apparatus used in connection with the method according to the present invention. It is also to be noted in this context that the sealing part or portion 4 may be formed as an integral part of the treatment cup 3, for instance.

In various embodiments, an opening for subjecting the target tissue such as skin to the suction may be defined by a treatment cup 3 (part or portion) of the treatment head 2. The treatment cup may be adaptable in size to best fit the shape of the treated body part. The adaptability may be implemented by a plurality of interchangeable cups of different size and/or by an adjustable cup. For example, the applicable size may range from about 10 or 20 mm to 80 or 90 mm in diameter depending on the dimensions and shape of the target area. As a general rule, a largest cup considered suitable for the area may be selected. For instance, 60-80 mm size may be more suitable for the neck than for facial areas that benefit from using a smaller diameter cup and related opening.

The treatment apparatus is thus not restricted to the examples of the figures in any way, but the apparatuses may be varied entirely freely within the scope of the claims. Thus, it is clear that the invention is by no means restricted to any specific shape or dimension of the treatment cup 3 or other components, for instance, but the shape and/or dimensions of the different elements and parts of the invention may differ from one another freely between embodiments, if desired. The idea of the present invention may even be applied in connection with such treatment heads at which rotating elements such as rollers are employed, as mentioned hereinbefore.

The low pressure in the low pressure chamber 6 is advantageously produced by using the aforementioned vacuum pump. Necessary adjusting valves are also advantageously mounted in connection with the vacuum pump.

In addition, in case an embodiment of the treatment apparatus with a central unit 8 is utilized, the hose 9 may have a valve which is advantageously positioned near the low pressure chamber 6. The speed of the system may further be improved by using the hose 9 as a low pressure reservoir. Thus, higher or lower pressure may be achieved in the hose 9 compared to the pressure desired in the low pressure chamber 6, especially in case a central unit 8 is utilized. If, for example, change of pressure from 150 to 200 mmHg is desired, the pressure of the low pressure chamber 6 is 150 mmHg and if in the subsequent phase a pressure of 200 mmHg is desired, the pressure of the hose 9 may already be set for example to 500 mmHg, so that upon opening of the valve a pressure of 200 mmHg is achieved quickly in the low pressure chamber 6 and the valve may be closed. The valve may be arranged to be controlled by pulse width modulation for adjusting the low pressure in the low pressure chamber 6. In some embodiments the valve may control higher frequency oscillation (described in more detail hereinafter) produced in addition to lower frequency suction pulses. Alternatively, higher frequency oscillation could be produced by other element(s), such as electric motor or ‘vibrator’. In some embodiments, the oscillation could be at least temporarily solely produced (i.e. no simultaneous lower frequency pulsation).

Subsequently, if a pressure of 150 mmHg is again desired, in which case a pressure of for example 50 mmHg may be set in the hose 9 so that a change from 200 mmHg to 150 mmHg is quick. Furthermore, the low pressure chamber 6 when positioned against the target area (skin) may be either substantially sealed or it may have a controlled leakage, for example, through a small opening.

The treatment apparatus utilizable in the method according to the present invention preferably comprises different sensors, one of which, for example, measures the composition of the skin tissue, such as the fluid content, fat content and/or oil content. Separate sensors may also be used for measuring fluid and fat contents. One sensor may, for example, measure the raised skin (bulge) produced by the suction effect and one other sensor may measure, for example, the suction force applied to the skin. The low pressure suction and suction force may be adjusted according to the results of the measurements. Therefore, the apparatus may be adaptive and especially dynamically adaptive. Yet, the treatment apparatus may comprise a sensor which measures the skin temperature.

Still, the apparatus may in some embodiments comprise a sensor for measuring the skin's blood circulation, the measurements of which may be used to adjust of the operation of the treatment apparatus. Further, the adjustment may be based on measurement of transepidermal water loss and skin pH.

Each sensor may be in connection with the treatment head 2 or to the treatment apparatus through a wired or wireless connection, such as, for example, a radio frequency signal, infrared signal or the like. Thus, the sensors may be an integrated or separate part of the treatment apparatus.

The apparatus may have at least one sensor that registers a signal given by the patient for increasing/decreasing the suction effect, based on which the adjustment of the suction may be done. The patient may thus give a signal to the sensor (for example, based on the pain experienced) and the sensor then relays to the apparatus the wish for the increase/decrease of suction efficiency. The sensor, such as a touch-registering sensor (e.g. a button), may be included in a user interface of the apparatus.

In one embodiment of the treatment apparatus, it may further comprise additional energy source(s) for warming the skin tissue and furthermore, means for automatically adjusting the energy source(s) to a set point value based on the measurements obtained by one or more sensors. Energy source may also be utilized to power up the apparatus for treating a patient.

Measurement techniques utilized in the embodiments of the treatment apparatus may include measurement of different sound frequencies, such as ultrasound and infrasound, techniques based on radiofrequencies and different wavelengths of light, i.e. optical measurement such as laser and infrared measurement, bioimpedance spectroscopy, magnetic resonance spectroscopy, Raman spectroscopy, nuclear magnetic resonance spectroscopy, microsensor mapping, heat camera imaging, or spectrofotometric intracutaneous imaging.

In some embodiments of the treatment apparatus, the potentially automated adjustment of the low pressure suction and/or suction force is based on mechanical characteristics and/or electrical characteristics and/or structure and/or composition of the skin. Mechanical characteristics include strength, flexibility, elasticity and resilience etc. Electrical characteristics include, for example, capacitance, impedance, resistance, reactance and inductance.

In addition, the potentially automated adjustment of the apparatus may in one embodiment be based on measurements of the flow of lymphatic fluid. The associated measuring techniques for the flow of lymphatic fluid may be selected from known techniques, such as, but not limiting to, isotope clearance technique.

In one embodiment of the treatment apparatus, the adjustment may also be based on the measurement of the patient's experience of cutaneous pain. Based on the experience, either the patient him/herself, or the operator, or both together adjust the apparatus's running parameters. Skin characteristics, when mentioned in this text, also include the pain felt and experienced on the skin.

In an embodiment of the treatment apparatus, a computer program guides in the application of the suction force by presenting the force level audibly and/or visually in the treatment head 2 and/or in the central unit 8 (e.g. via display 10 in case of visual information). Low pressure suction may be adjusted automatically using the computer program, and thus it is not necessary for the patient or operator to adjust the low pressure suction during the treatment. Advantageously, when the treatment force exceeds the permitted value, the program stops the apparatus or lowers the suction force.

The computer program may be utilized to calculate the target value of one or more on-going treatment forces, such as suction force, based on the measurements obtained and/or on the desired value of the suction pressure. Therefore, the apparatus also comprises sensor/sensors as mentioned hereinbefore for measuring one or more ongoing treatment forces, such as the level of the suction force. A computer program may be configured to automatically calculate and adjust the level of low pressure suction to the target value, based on the measurements obtained. The parameters/results of the measurements, which may be taken into account in determining the target value of the low pressure suction, include e.g. fluid content of the skin tissue, fat content of the skin tissue, the bulge i.e. the lift of the skin tissue (the size of the fold in the skin) and/or the skin temperature.

Furthermore, the computer program may be functionally connected to database, which contains the patient's treatment information. The database may be remote and hosted by a remote computer or computer system, which is accessed via a communications connection or network, e.g. the Internet. For the purpose the treatment may include a wired or wireless data interface, e.g. USB (Universal Serial Bus), Bluetooth™, NFC/RFID (Near-Field Communication/Radio Frequency Identification), cellular, wireless LAN (Local Area network) or wired LAN interface.

In one embodiment of the treatment apparatus, it is desired to combine slow, pulsating low pressure to a faster impulse-like oscillation treatment. For instance, the oscillation treatment may be modulated by a pulsating low pressure treatment.

In one embodiment of the treatment apparatus, the treatment apparatus is arranged to provide to the low pressure chamber 6 simultaneously a pulsating low pressure treatment, which preferably has a frequency of below 5 Hz, and an oscillation treatment, which for its part, preferably has a frequency of more than 5 Hz. In other embodiment, the threshold frequency between low pressure pulses and high frequency oscillation may be different.

Moreover, a high frequency oscillation treatment may be particularly added to the suction phase of the low frequency pulsating low pressure treatment. The oscillation may extend over the whole duration of the low pressure pulse. In some embodiments, in addition to the duration of the pulse, the oscillation could be present also during the neutral portion of the signal period. In some embodiments, the duty cycle of 100% (i.e. continuous suction/pulse) may be applied, whereupon also the optional oscillation may be on all the time. High frequency in this instance may mean for example an impulse-like pressure change or oscillation with a frequency of more than 5, 10, 15 Hz or higher frequencies such as e.g. 90 Hz or even as high as 200 Hz. The hose 9 may be arranged in this case to be used as a pressure reservoir for accelerating pressure variations in the low pressure chamber 6.

In an embodiment, the pulsation frequency may range from about 0.1 Hz to about 5 Hz, preferably at least to about 3.5 Hz, or occasionally even up to 10 Hz depending on the particular embodiment of the apparatus (supported frequencies). For instance, it may be about 0.5 Hz that corresponds to a 2 second signal period and 1 second pulse duration with 50/50 duty cycle. The suction on the skin tissue when the treatment cup 3 is facing the skin is naturally introduced during the associated on-time (i.e. 1 second in case of 2 second total signal period with 50/50 duty cycle).

Several techniques, duty cycles, pressures and frequencies may be used for different conditions causing the neuropathic pain. Continuous (duty cycle 100%) suction technique can be especially useful when the purpose is to ‘make room’ for nerves and especially for the nerve ends which are compressed or entrapped by, e.g., a fascia, and thus cause neuropathic pain. Continuous low pressure suction technique is, advantageously, combined with the high frequency oscillation treatment. The high frequency, preferably in the range of 20-90 Hz, may be chosen/adjusted so that the penetration of the treatment effect can be varied in order to reach proper depths of the skin tissue in which the fascia resides. Value of the high frequency oscillation of 60 Hz may be used for treatment targeted to a superficial fascia, for instance.

If the high frequency oscillation treatment is used in treating neuropathic pain, typically, higher values for the low pressure may be used such as, e.g., 100-130 mmHg compared to 50-80 mmHg without the high frequency oscillation treatment. If there is fibrotic tissue on the target area, the low pressure value used may be as high as, e.g. 250 mmHg, advantageously in conjunction with the high frequency oscillation treatment, preferably in the range of 60-90 Hz. Preferably the configured frequency is user-adjustable via a user interface of the apparatus. The user interface may include a number of control input elements in the form of a touch display, touch pad, button, mouse, Scrollpoint™, roller, voice input interface, keypad, etc. for the purpose.

Accordingly, the UI (user interface) may include e.g. a display and/or audio response interface (typically buzzer or loudspeaker) for data visualization and feedback provision towards the apparatus operator. The operator may be the patient himself/herself or other person who preferably has adequate medical and technical skills to operate the apparatus in sufficient fashion.

Typical pressure (suction, i.e. negative pressure) of the treatment apparatus may preferably be of the order of about 80 mmHg, falling e.g. within a range from about 5, 10 or 30 to about 250 mmHg, or even up to about 350 mmHg or higher, e.g. about 500 mmHg. As being clear based on this disclosure, the negative pressure is preferably user-adjustable or -selectable in at least most embodiments. However, use of fixed (user non-adjustable) pressure and potentially other fixed parameters is possible in some embodiments of the apparatus as well. Such embodiments could be targeted to certain very specific use scenarios or applications, for example.

In some embodiments, the operator of the device is provided, via the UI of the device, a pressure setting and/or pressure readings in predetermined, optionally user-selectable, units such as mmHg or pascal. Alternatively or additionally, e.g. a numeric value in a predetermined scale, e.g. between one and five or one and ten, without any particular units could be used for adjustments and/or indicated to the operator. One end of the scale could represent predetermined minimum suction or zero suction, whereas the other end (e.g. maximum number) could represent predetermined maximum suction. The apparatus manages the conversion between the user-indicated pressure and corresponding real pressure established.

Instead or in addition to numeric values, the pressure and/or other parameters could be indicated through other symbols, optionally using dot/circle, star, line, curve or rectangular shapes.

The order of magnitude of high frequency oscillation may range, for example, from about 2 to about 200 Hz, preferably at least from about 5 or 10 Hz to e.g. about 100 Hz.

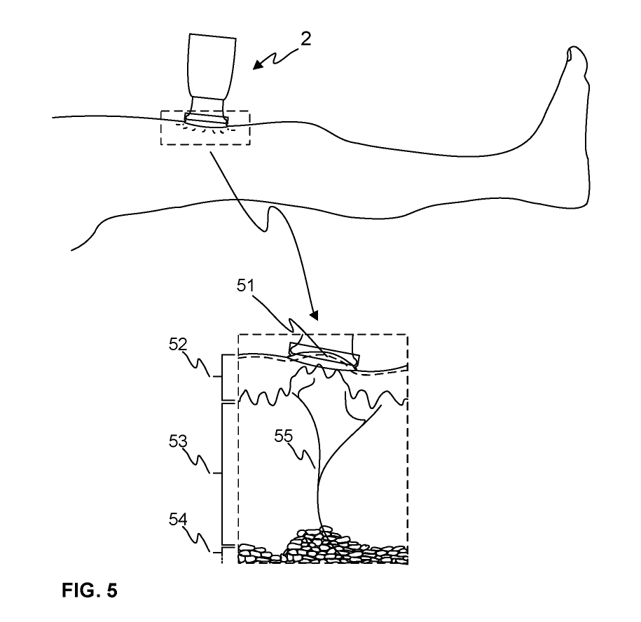

FIG. 5 thus depicts schematically a use scenario of low pressure suction apparatus in treating, in particular, at an area of leg of a patient. The patient may sit or stand in upright position during the treatment, for instance. The treatment head 2 is pressed towards the skin tissue on the leg. The low pressure suction, i.e. negative pressure flow, causes the tissue to rise into the treatment cup, thus causing a bulge 51 to the skin tissue. The lower layers, such as e.g. epidermis 52, dermis 53 and hypodermis 54, of the skin tissue are simultaneously mobilized. The figure is a simplified illustration of the skin tissue, neglecting e.g. hairs, hair follicles, sweat glands, blood and lymphatic vessels, etc. The mobilization of the tissue can ‘make room’ for nerves 55. This can relieve the pressure produced by e.g. fascia on the nerve. Neuropathic pain may be alleviated by mobilizing the tissue around the nerves by using the low pressure suction, thus decreasing the pressure on the nerves. In case of lymphatic vessels, low pressure suction activates the lymphatic system and facilitates movement of the lymph in the lymphatic vessels thus carrying e.g. inflammatory agents away from the tissue.

Optionally, lubricant such as massage oil may be initially smeared on a target area of the skin. Care shall be taken that the lubricant does not contain particles that could end up within the apparatus during the treatment to avoid clogging the internals thereof and related cleaning procedures.

The treatment head is located so that the associated contact portion, such as preferably replaceable treatment cup, is in close contact with the skin area to be treated, e.g. on the quadriceps (shown), spine, or e.g. sternum.

The diameter of an optimum cup generally varies between patients and from a treating technique to another, but in standard case it may range from about 60 mm to about 80 mm, for example. As an applicable basic rule, one could consider to select the largest suitable treating cup for each target area. Fastening of the treatment cup having regard to the rest of the treatment head may incorporate grooves (in the cup or head) and matching lips (in the head or cup, respectively), snap fastener(s), threads, magnets, frictional and/or pressure contact (e.g. based on the elasticity (enabling stretching) and/or roughness of the contacting surface(s) yielding tight, secure fit), or any combination of the above or other feasible attachment technologies providing e.g. sufficiently secure and airtight fit between the connected elements.

The treatment head/cup should be hold onto relatively lightly. The cup may typically be kept on the same location for about three to five pulsations, whereafter it may be moved to adjacent skin area with e.g. ⅓ overlap. The total number of locations, or spots, that are treated depends on the overall coverage of the treated condition, which typically defines the target area of treatment, as well as the size of the cup and related suction opening. The area may encompass from about one or two to ten locations, for instance.

Either stationary, sliding or hybrid technique may be applied having regard to the lifting of the treatment head during the movement thereof on the skin between the different treated areas. The general direction of motion may be sideways and/or from the top to the bottom (i.e. from the head towards the torso or shoulder line). Thus the overall duration of treating a certain area or spot at a time commonly ranges from about one or few seconds to few tens of seconds depending on the utilized pulse duration and duty cycle and thus the overall signal period.

Pulse length/duration may be about one second, for example, and the used pressure between about 5-350 mmHg, advantageously from about 20 mmHg to 130 mmHg. Duty cycle may be about 50/50 (50%) between the pulse period and passive period, i.e. pulse-containing and neutral portions of the treatment signal, respectively, whereupon the overall repeating signal period comprising the pulse portion and neutral/passive portion covers two seconds. The duty cycle with two-second signal period may also be, e.g. 20/80 (20%) or 30/70 (30%) in which cases the pulse-containing portion lasts 0.4 or 0.6 seconds and neutral portion 1.6 or 1.4 seconds, respectively. The signal period may be varied preferably from a half to five seconds thus meaning pulsation frequency of 0.2 to 2 Hz. The high frequency oscillation additionally provided at least or exclusively during the pulse-containing period (1 second in case of duty cycle of 50/50 (50%) with a two-second signal period) may preferably be from 20-90 Hz.

One shall acknowledge the fact that also the neutral period (at least no low frequency suction) is usually treatment-wise important e.g. in a sense that during it the skin stretching stops and the skin recovers its relaxed position. The effectiveness of the treatment is in many respects due to the back-and-forth movement of the skin, not just due to suction-based stretching thereof.

Having regard to various embodiments, typically one therapy session lasts for about 10-60 minutes at a time. A treatment period may include multiple sessions, e.g. about 10 sessions. As mentioned hereinbefore, in many cases the obtained results are not necessarily permanent, whereupon the therapy should be regularly practiced even after a more intensive therapy period, e.g. once a week.

Considering the general direction of motion of the treatment head in the therapy sessions, one could conclude that typically the locations closer to the two subclavian veins may be treated prior to locations farther away therefrom, e.g. limb extremes, so that the main lymphatic channels are opened for the lymphatic flow first instead of trying to open the extreme conduits first while the flow is still blocked centrally.

In some cases, such as e.g., in chemotherapy-induced neuropathy, which typically manifests itself as neuropathic pain in the body extremities (hands and feet), the use of low pressure suction to said extremities to activate lymphatic system may at least ameliorate, even remove, the pain experienced by the patient. Alleviation of the pain may be due to the increased circulation of blood and lymph by using the low pressure suction in the extremities. Therefore, in case of moving the lymph towards the heart, it may happen that the waste including chemotherapy related chemical substances are removed from the tissues of the extremities which may alleviate the neuropathic pain. Pressure values used for hands and feet may typically be high, e.g. about 250 mmHg. The low pressure may, preferably, be applied in continuous manner without pulsation. One treatment session may take from about 3 to about 5 minutes per hand and/or feet, for example. These treatment sessions may be repeated, for example, two times a week for 4 to 12 weeks, or advantageously, for 6 to 8 weeks.

In some cases, the low pressure suction treatment may also be used to alleviate pain resembling neuropathic pain, or in some instances also classified as neuropathic pain, such as e.g. in case of fibromyalgia which is characterized by chronic widespread pain. Low pressure suction treatment may also be beneficial in other cases which typically cause neurological symptoms such as e.g. Lyme disease. Low pressure suction treatment, advantageously in the early stage of Lyme disease, preferably together with antibiotic treatment, may ameliorate or even cure the patient. This may be due to enhancement of the immune response when using low pressure suction to increase circulation and to activate lymphatic system.

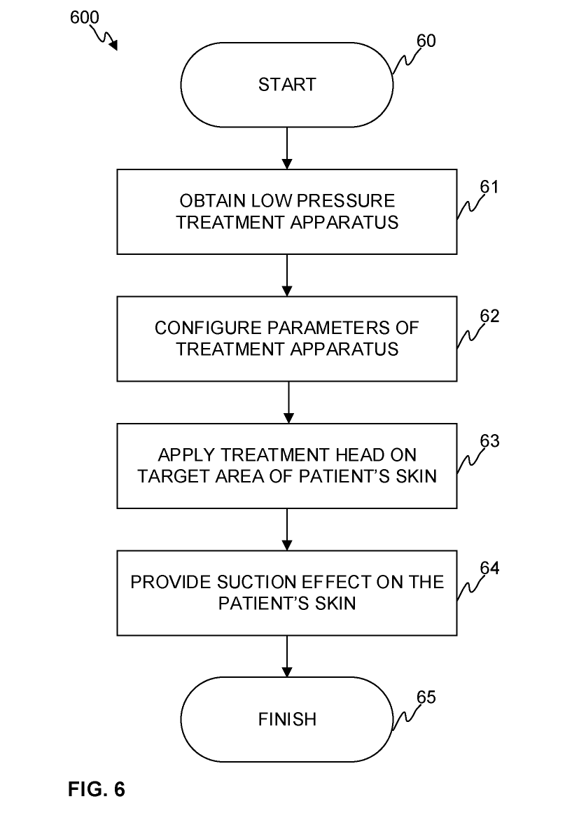

FIG. 6 is a flow diagram, at 600, disclosing an embodiment of a method in accordance with the present invention.

Item 60 refers to a start-up phase. Decision to treat a patient in accordance with the principles of the present invention is made.

At 61, an embodiment of a treatment apparatus is obtained. It may be purchased, borrowed or rented, for example. Further gear, such as massage oil and treatment chair or table/platform may optionally be further acquired at this stage.

At 62, the apparatus and possible other equipment (e.g. chair/table) are configured, which may refer to adjusting, via the UI of the apparatus, desired parameters for the treatment including e.g. suction pressure, pulsation frequency, oscillation frequency, duty cycle, etc. A patient may himself/herself configure and subsequently utilize the apparatus. Alternatively, the apparatus may be operated by some other party, such as a professional operator such as a medical professional, a therapist, a nurse, a friend or a family member of the patient. Already for the configuration tasks, the apparatus may have to be turned on unless the configuration can be purely adjusted by using e.g. mechanical switches, sliders or other elements that continuously remember their state in contrast to e.g. touch pads or touch displays that have to be powered up first to register the user input.

At 63, the treatment head is (re-)positioned on a target area or sub-area thereof to be treated, which usually involves placing the head, or in practice the contact part of the associated treatment cup or similar element, in contact with the skin of the patient. Depending on the nature of the treatment, stationary, sliding or hybrid application technique may be selected.

At 64, the treatment is executed having regard to the target area by subjecting the area to the low pressure (suction) pulses and intervening neutral or passive periods.

In practice, items 63 and 64 are usually simultaneously and/or sequentially repeatedly executed during a treatment session. Accordingly, their mutual execution order may be considered to vary. The treatment head/cup at a certain location usually covers only a small sub-area of the overall target area at a time, whereupon the head shall be moved along a desired route on the skin to cover the target area in its entirety using a preferred application technique.

Method execution is ended at 65. The suction and the apparatus in general may be turned off. Alternatively, at least some measurement results and/or other gathered statistics concerning the treatment, e.g. pressure, pulse characteristics, or various durations, may be inspected via a display or other feasible element of the treatment apparatus itself, or outputted therefrom via an available data communication interface either wirelessly or wiredly. The data may be transferred to a near-by device or a remote system, optionally via the Internet, for storage, inspection (e.g. visualization) and/or analysis. The external device or system may host a database for storing data from a number of treatment apparatuses. The patient may be instructed to drink water to prevent dehydration.

In some variations of the treatment apparatus described herein, the treatment head and related elements, e.g. suction cups thereof, could be configured for enabling substantially contactless operation in addition to or instead of contact-based therapy. During the contactless therapy, the treatment head could be merely hovered close to the target area without actually contacting the skin, for instance. The apparatus could be provided with audible, tactile and/or visible guidance element such as loudspeaker, buzzer, vibration element, indicative lamps (e.g. LEDs) and/or a display, optionally touchscreen, The guidance element could indicate, in real-time fashion, current and/or proper distance between the target surface (e.g. skin) and the treatment head. In contact-based treatment, the cup/flexible element of the treatment head inherently provides such guidance for maintaining a proper distance (in that case, contact) between the head and target surface.

1. A method for at least temporarily alleviating neuropathic pain, such as reducing pain in patients with a diabetic neuropathy, chemotherapy-induced neuropathy, fibromyalgia or Lyme disease, comprising
obtaining a preferably both electrically powered and portable treatment apparatus capable of contacting, via a treatment head, a target area of a patient and introducing low-pressure suction, i.e. negative pressure flow, thereto,
configuring the treatment apparatus so as to introduce predefined pulsating pressure variation or, preferably, continuous low pressure suction, i.e. negative pressure flow, to a suction opening arranged at the treatment head, and
applying the suction opening of the treatment head on the target area while the treatment apparatus is on and providing the suction effect corresponding to the configuration.

2. The method of claim 1, wherein the treatment head comprises a preferably replaceable treatment cup for contacting the skin of the patient, preferably without substantial clearance, further wherein a proper cup is selected from a plurality of cups responsive to the characteristics and/or location of the target area to be treated.

3. The method of claim 1, wherein the diameter of the suction opening of the head, preferably of the cup thereat, falls within a range of about 10 mm to about 85 mm.

4. The method of claim 1, wherein the pulsating pressure variation comprises low pressure pulses substantially in the range from about half a second to two seconds, preferably about one second, in duration and/or from about 5 mmHg to 350 mmHg, advantageously from about 20 mmHg to 130 mmHg in strength.

5. The method of claim 1, wherein an overall signal period that is then repeated contains a pulse period with suction effect and temporally adjacent passive period substantially introducing no suction, wherein the relationship between the two is optionally determined by a preferably user-adjustable duty cycle parameter of the apparatus.

6. The method of claim 1, wherein the pulsating pressure variation includes, in addition to low frequency pulses, simultaneous higher frequency oscillation, preferably falling within a range of about 5 to about 100 Hz.

7. The method of claim 1, wherein the treating head is maintained at a certain location of the target area for a duration covering about 3-5 pulses at a time.

8. The method of claim 1, wherein two successively treated locations of the target area overlap, preferably about 20-50%.

9. The method of claim 1, wherein a number of same locations are alternately treated during a single treatment session.

10. The method of claim 1, wherein the application technique of the head is, in terms of motion, stationary, sliding or a hybrid between the two involving maintaining the head at a certain location for some time while also sliding the head substantially in skin contact between locations.

11. The method of claim 1, wherein lubricant, optionally massage oil, is initially applied to the target area.

12. The method of claim 1, wherein the apparatus comprises a central unit connected to the treatment head at least by a hose.

13. The method of claim 1, spanning several treatment sessions distributed over time, optionally substantially regular sessions or at least about 5-15 sessions, preferably of at least about 10 minutes in duration and/or at least about once a week.

14. An electrically powered, portable treatment apparatus for at least temporarily alleviating neuropathic pain, such as reducing pain in patients with a diabetic neuropathy, chemotherapy-induced neuropathy, fibromyalgia or Lyme disease, wherein the apparatus comprises a treatment head configured to contact a target area of a patient and to introduce low-pressure suction thereto, and
a low pressure generating element operatively coupled to the treatment head controlled by a processing unit so as to introduce predefined, advantageously, pulsating pressure variation to a suction opening arranged at the treatment head.

15. The electrically powered, portable treatment apparatus of claim 14, wherein the diameter of the suction opening of the head, preferably of the cup thereat, falls within a range of about 10 mm to about 85 mm.

16. The electrically powered, portable treatment apparatus of claim 14, wherein the processing unit is configured to cause the apparatus to produce the pulsating pressure variation comprising low pressure pulses substantially in the range from about half a second to two seconds, preferably about one second, in duration and/or from about 5 mmHg to 350 mmHg, advantageously from about 20 mmHg to 130 mmHg, in strength.

17. The electrically powered, portable treatment apparatus of claim 14, wherein the processing unit is configured to cause the apparatus to produce the pulsating pressure variation comprising, in addition to low frequency pulses, simultaneous higher frequency oscillation, preferably falling within a range of about 5 to about 100 Hz.
